(19) United States
(12) Patent Application Publication
(10) Pub. No.: US 2008/0050724 A1
(43) Pub. Date: Feb. 28, 2008

(54) Method of detecting one or more limited copy targets

(75) Inventor: Amy J. Devitt, San Carlos, CA (US)

Correspondence Address: HAVERSTOCK & OWENS LLP, 162 N WOLFE ROAD, SUNNYVALE, CA, 94086, US

(73) Assignee: MicroFluidic Systems, Inc.

(21) Appl. No.: 11/509,868

(22) Filed: Aug. 24, 2006

(51) Int. Cl. C12Q 1/68

(52) U.S. Cl. 435/6

(57) ABSTRACT

A method allowing simultaneous amplification of multiple low-abundance targets in environmental samples. This is a two-step process that includes a combined reverse transcription and pre-amplification step, which utilizes a mix of gene-specific primer sets, followed by a second amplification step performed on the previously generated “RT-amplification” product. Initial amplification of each target is performed prior to the splitting of the sample for individual amplification and identification. The method combines the process of reverse transcription and amplification within a single processing apparatus. The method also enables gene-specific reverse transcription using gene-specific primers, thereby reducing if not eliminating non-specific product in this reverse transcription step of the process.

### FIELD OF THE INVENTION

The invention relates to a method of detecting the presence of low copy targets within a sample. More particularly, the invention relates to a method of detecting the presence of multiple different types of low copy nucleic acids within a sample.

### BACKGROUND OF THE INVENTION

Polymerase chain reaction (PCR) is a molecular technique for enzymatically replicating specific DNA sequences. In particular, PCR is used to amplify relatively short, well-defined nucleotide sequences within a given DNA strand. A specific DNA sequence to be amplified is determined by selecting primers. Primers are short, artificial DNA strands, often not more than fifty and usually only 18 to 25 base pairs long that are complementary to the beginning and end of the specific DNA sequence to be amplified. The primers bond to the DNA strand at these starting and ending points and begin the synthesis of the new DNA strand.

FIG. 1 illustrates a conventional method of amplifying and detecting a specific low copy DNA sequence within a sample. In order to detect a specific sequence of a low copy DNA strand, the DNA within the sample must be amplified. In its original low copy state, the DNA sequence does not include sufficient quantity as to be detectable. In the step 5, a sample is provided that includes a low copy DNA strand. The objective is to detect if a specific DNA sequence is present within the sample. At the step 10, reagents and a first pair of DNA primers specific to the DNA sequence to be amplified and detected are added to the sample solution. Each of the primers is configured to bond with the DNA strand such that the desired DNA sequence is bound at either end by the primer pair. At the step 15, a first amplification step is performed on the sample solution. Typically, amplification is performed by thermal cycling, or PCR. Each thermal cycle is considered a heating step and a cooling step. The heating step is also referred to as a denaturation step. The cooling step is also referred to as an annealing and extension step. The maximum number of thermal cycles are performed without generating non-specific product. In most cases, the maximum number of thermal cycles that can be performed without generating non-specific product is between about 40 and 45 thermal cycles. Performing more thermal cycles than this maximum will result in additional sample copies; however, the probability of non-specific products increases thereby decreasing the confidence that any detected signal is valid.

At the step 20, additional reagents and a second pair of DNA primers are added to the previously amplified sample solution from the step 15. The reagents typically include detection chemistries, such as TaqMan® probes. Each of the second pair of primers is internal to the first pair of primers, referred to as nested PCR. In most applications, the nucleotide sequence of each of the second pair of primers does not overlap with the nucleotide sequences of each of the first pair so as to avoid generation of non-specific products. In some applications however, there is some overlap between the nucleotide sequences of each of the first and second pairs of primers. At the step 25, a second amplification step is performed on the previously amplified sample solution. As with the first amplification step, the second amplification step is typically performed by thermal cycling. Up to 40 thermal cycles are performed, and in some applications, up to 45 thermal cycles. The result is a sample solution including an amplified number of DNA strands corresponding to the specific DNA sequence. At the step 30, the amplified DNA strands are detected. Typically, during the amplification steps the detection chemistries are stimulated, such as releasing a detectable flourescent probe, which are then detected using any number of conventional detection means.

Two separate amplification steps are performed because the reagents become depleted after a certain number of thermal cycles. Therefore, after the first amplification step the amplified sample solution is diluted with additional reagents to enable additional amplification during the second amplification step.

The objective of the amplification and detection method described in relation to FIG. 1 is to detect the presence of a given DNA sequence. The method is not useful in determining the actual number of specific DNA sequences, or the number of the specific DNA sequence relative to other specific DNA sequences. The method is also ineffective when applied to an RNA sample and determining the relative number of specific RNA sequences relative to other specific RNA sequences, such as in gene expression applications.

FIG. 2 illustrates a conventional method of quantifying the relative number of specific RNA sequences within a sample. The method of FIG. 2 includes a reverse transcription and polymerase chain reaction (RT-PCR) process. The specific RNA sequences are typically low copy and therefore need to be amplified in order to be detected. To detect a specific sequence of a low copy RNA strand, all RNA strands in the original sample are first reverse transcribed into corresponding DNA strands. In this manner, the RNA-specific sequences to be detected are reverse transcribed, but so too are all other RNA sequences present in the original sample. For multiple different RNA strands, each different RNA strand is reverse transcribed into a corresponding DNA strand. When reverse transcribed from a low copy RNA strand, the DNA strand and any specific DNA sequences do not include sufficient quantity as to be detectable, and therefore require amplification in a manner similar to that described in relation to FIG. 1. At the step 50, a sample is provided that includes multiple low copy RNA strands. The objective is to quantify the relative number of each specific RNA sequence. At the step 55, a reverse transcription process is performed on the sample such that all RNA strands included in the sample are reverse transcribed into complimentary DNA strands. In this manner, non-specific DNA strands are generated in addition to any specific DNA sequences corresponding to the specific RNA sequences to be quantified. Reverse transcription is conventionally performed in an incubation chamber. Once the reverse transcription process is completed, the sample solution is removed from the incubation chamber and placed in a thermal cycling chamber.

At the step 60, reagents and a first pair of DNA primers are added to the sample solution. At the step 65, a linear phase amplification step is performed on the sample solution. Linear phase amplification is performed by thermal cycling. Linear phase amplification is that portion of the amplification process that maintains a relative number of each different DNA-specific sequence present. That is, during linear phase amplification each different DNA-specific sequence is amplified at the same rate, thereby maintaining the relative difference in numbers. Typically, the linear phase is maintained for 5-15 thermal cycles.

At the step 70, additional reagents and a second pair of DNA primers are added to the first amplified sample solution. The reagents typically include detection chemistries. Each of the second pair of primers is internal to the first pair of primers. At the step 75, a second amplification step is performed on the first amplified sample solution. As with the amplification step performed at the step 65, the second amplification step is typically performed by thermal cycling. Up to 40 thermal cycles are performed, and in some applications, up to 45 thermal cycles. The result is a sample solution including amplified numbers of multiple specific DNA sequences. In theory, the relative difference in the number of each amplified DNA-specific sequence is the same as the original RNA sample. At the step 80, the amplified DNA-specific sequences are detected. Typically, during the second amplification step the detection chemistries are stimulated, such as releasing a specific detectable flourescent probe for each specific DNA sequence, which are then detected using any number of conventional detection means. The intensity of each probe is also detected, thereby determining the quantity of each specific DNA sequence. This method can also be used to detect the presence of multiple different RNA-specific sequences without determining the relative quantity of each specific RNA sequence.

Detecting the presence of specific RNA sequences or specific DNA sequences has varied applications, such as bio-threat detection. Since very low levels of bio-agent can have a serious impact, detecting threats in the environment requires very aggressive limit of detection requirements. However, current detection methods including TaqMan® and DNA microarrays require relatively large amounts of starting material (1-10 ug) and therefore cannot be applied directly to a biosensor. In addition, hybridization-based microarray methods have lower sensitivity and specificity compared with PCR-based approaches. RT-PCR has the highest specificity and sensitivity among all available methods, although this approach has low throughput and relatively large amounts of starting RNA are required.

### SUMMARY OF THE INVENTION

Embodiments of the present invention are directed to a method that allows simultaneous amplification of multiple low-abundance targets in environmental samples. This is a two-step process that includes a combined reverse transcription and first amplification step, which utilizes a mix of gene-specific primers, followed by a second amplification step performed on the product generated during the reverse transcription and first amplification step. In some embodiments, each amplification step is performed using PCR. Reagents added for the second amplification step can include any conventional detection chemistries, such as nested TaqMan® primers and probes.

Purified RNA and/or DNA are reverse transcribed with a multiplex of gene-specific primers and amplified for a determined number of thermal cycles in a one-step combined RT-PCR reaction. Amplification via PCR is performed on small aliquots of generated “RT-amplification” product with nested primers in individual, or less multiplexed reactions. The reliability of detection depends on the initial number of copies in the PCR; therefore, statistically it is more favorable to conduct the combined RT-PCR amplification on a whole rather than on a split sample. This allows an initial amplification of up to 1000 fold (depending on the number of thermal cycles performed in the first amplification step) of the existing copies of target prior to the splitting of the sample for individual amplification and identification. This greatly increases the chance that sufficient amount of target is available to be amplified, if the target is present in the original sample.

In addition to amplifying the targets prior to splitting the sample, the method combines the process of a reverse transcription step and first amplification step within a single processing apparatus, thereby eliminating the need to transfer the sample from an incubation chamber to a thermal cycling chamber. The method also enables gene-specific reverse transcription using gene-specific primers. In this manner, one or more specific gene sequences are reverse transcribed, thereby reducing if not eliminating non-specific product in this reverse transcription step of the process.

The amplification and detection method can be used in any application where it is necessary to identify one or more targets from a limited sample, particularly where a target is a low copy target. This method is useful for unknown samples that may include a number of targets, such as for diagnostic applications and screening samples for potential bio-threat agents.

In some embodiments, the amplification and detection method is implemented within an amplification and detection apparatus. The amplification and detection apparatus includes an amplification apparatus and a detection apparatus integrated within a single device. Alternatively, the detection apparatus is configured as a stand-alone device and is coupled to the amplification apparatus. The amplification and detection apparatus can be configured to automatically perform one, some, or all of the steps of the amplification and detection method. In this manner, one, some, or all of the steps of the amplification and detection method can be automated.

### BRIEF DESCRIPTION OF THE DRAWINGS

FIG. 1 illustrates a conventional method of amplifying and detecting a specific low copy DNA sequence within a sample.

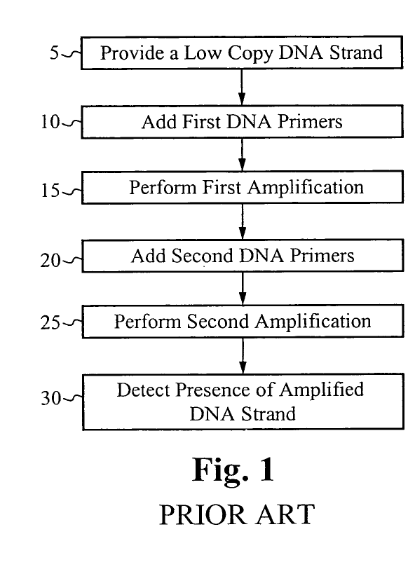

FIG. 2 illustrates a conventional method of quantifying the relative number of specific RNA sequences within a sample.

FIG. 3 illustrates a first amplification and detection method for detecting the presence of a low copy target.

FIG. 4 illustrates a second amplification and detection method for detecting the presence of multiple different low copy targets.

FIG. 5 illustrates a third amplification and detection method for detecting the presence of a low copy target.

FIG. 6 illustrates a fourth amplification and detection method for detecting the presence of multiple different low copy targets.

FIG. 7 illustrates a functional block diagram of an exemplary amplification and detection apparatus.

Embodiments of the amplification and detection method and apparatus are described relative to the several views of the drawings. Where appropriate and only where identical elements are disclosed and shown in more than one drawing, the same reference numeral will be used to represent such identical elements.

### DETAILED DESCRIPTION OF THE PRESENT INVENTION

Embodiments of the amplification and detection method include a reverse transcription step in which select gene sequences are reverse transcribed, thereby reducing if not eliminating the reverse transcription of non-specific products, that is gene sequences that are not to be detected. The reverse transcribed gene-specific sequences are then amplified and detected. The amplification and detection method can be simultaneously applied to multiple different gene-specific sequences. For simplicity, the amplification and detection method is described in terms of reverse transcribing gene-specific RNA sequences into corresponding gene-specific cDNA sequences, and then amplifying the gene-specific cDNA sequences. It is understood that the amplification and detection method is applicable to any type of gene sequence.

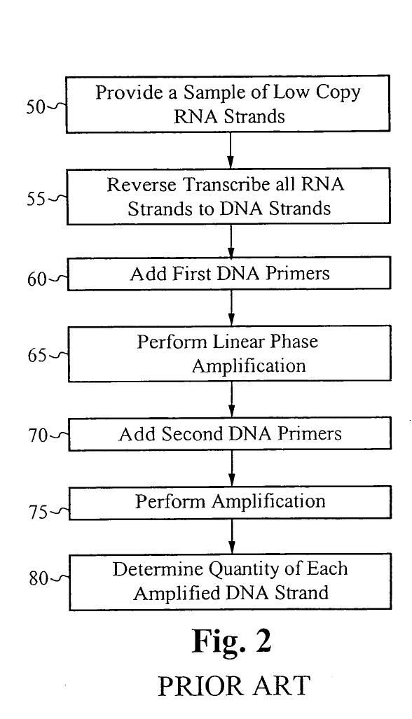

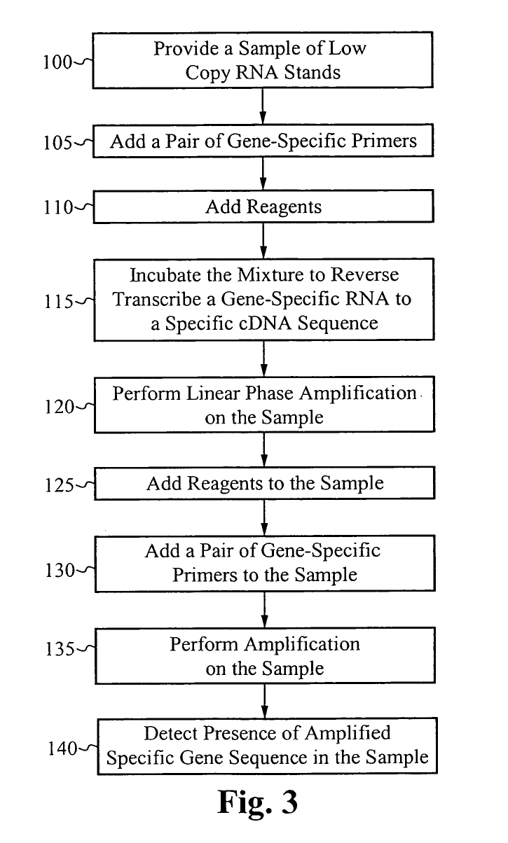

FIG. 3 illustrates a first amplification and detection method for detecting the presence of a low copy target. In some embodiments, a target is a specific gene sequence, such as a gene-specific DNA sequence or a gene-specific RNA sequence. The method of FIG. 3 includes a reverse transcription and polymerase chain reaction (RT-PCR) process. However, unlike the RT-PCR process described in relation to FIG. 2 in which all RNA strands present in the original sample are reverse transcribed, only a specific gene sequence in the RNA is reverse transcribed in the method of FIG. 3. This reduces, if not eliminates, the reverse transcription of non-specific products and reduces, if not eliminates, competition for the amplification reagents used in a subsequent first amplification step. This improves the efficiency of the amplification process. The specific gene sequence to be detected is typically low copy and typically does not include sufficient quantity as to be detectable. Therefore, after the reverse transcription from the gene-specific sequence to a corresponding gene-specific cDNA, the gene-specific cDNA is amplified in order to be detected.

At the step 100, a sample is provided that includes a low copy RNA strand. An objective is to detect if a specific gene sequence in the RNA is present within the sample. At the step 105 a pair of gene-specific primers are added to the sample. By way of example, the specific gene sequence that is to be targeted and amplified is referred to as gene 1. In this case, the pair of gene-specific primers are specific to gene 1. One of the pair of gene-specific primers, referred to in this case as the gene 1 reverse transcription (RT) primer, is configured to bond with the RNA strand at one end of the gene. At the step 110, reagents are added to the sample. It is understood that the step 105 and the step 110 can be combined as a single step. The amplification and detection method combines a reverse transcription step and a first amplification step within a single processing chamber. The pair of gene-specific primers including the gene 1 RT primer, reagents for the reverse transcription step, and reagents for the first amplification step are all added to the original sample prior to performing the reverse transcription step. At the step 115, the mixture including the sample, the pair of gene-specific primers, and the reagents, is incubated at a first temperature for a first period of time. During this incubation period, the gene 1 RT primer binds to an RNA strand if the RNA strand includes the gene 1. The bound gene 1 RT primer initiates the reverse transcription of the gene 1 in the RNA to a corresponding cDNA sequence, including the gene 1. During this incubation step, the gene-specific sequence in the RNA is identified and reverse transcribed into the corresponding specific cDNA sequence.

After the first period of time, a first amplification step is performed on the reverse transcribed sample solution at the step 120. The first amplification step is performed by thermal cycling, or PCR. In the first amplification and detection method, the first amplification step is limited to the linear phase. Linear phase amplification is maintained during about 5-15 thermal cycles. In some embodiments, the incubation step in which reverse transcription takes place and the first amplification step are performed in a single processing chamber. Since the pair of gene-specific primers and the reagents needed for reverse transcription and the first amplification are previously added, there is no need to add reagents and/or additional primers to the sample solution in between the incubation step and the first amplification step.

At the step 125, additional reagents are added to the first amplified sample solution, thereby diluting the first amplified sample solution. In some embodiments, an additional dilution step is performed whereby an additional neutral solution, such as water, is added in addition to the reagents added in the step 125. The reagents include detection chemistries, such as TaqMan® probes. At the step 130, a pair of gene-specific primers targeted to the gene 1 is added to the first amplified sample solution. The pair of gene-specific primers added at the step 130 can be the same as the pair of gene-specific primers added at the step 105. Alternatively, the pair of gene-specific primers added at the step 130 are not the same as the pair of gene-specific primers added at the step 105. Instead, each of the pair of gene-specific primers added in the step 130 are internal to the pair of gene-specific primers added at the step 105. In general, the pair of gene-specific primers added at the step 130 is equal to or internal to the pair of gene-specific primers added at the step 105. At the step 135, a second amplification step is performed on the first amplified sample solution. As with the first amplification step, the second amplification step is performed by thermal cycling. In some embodiments, up to 40 thermal cycles are performed. Alternatively, more than 40 thermal cycles are performed. In general, the number of thermal cycles performed during the second amplification step is sufficient for entering into an exponential phase amplification and reaching saturation. Saturation is the point where additional cycles do not increase the yield of specific product. The result is a sample solution including an amplified number of cDNA strands including the gene-specific sequence. At the step 140, the amplified specific gene sequence is detected, if present. Typically, during the second amplification step the detection chemistries are stimulated, such as releasing a detectable flourescent probe, which are then detected using any number of conventional detection means.

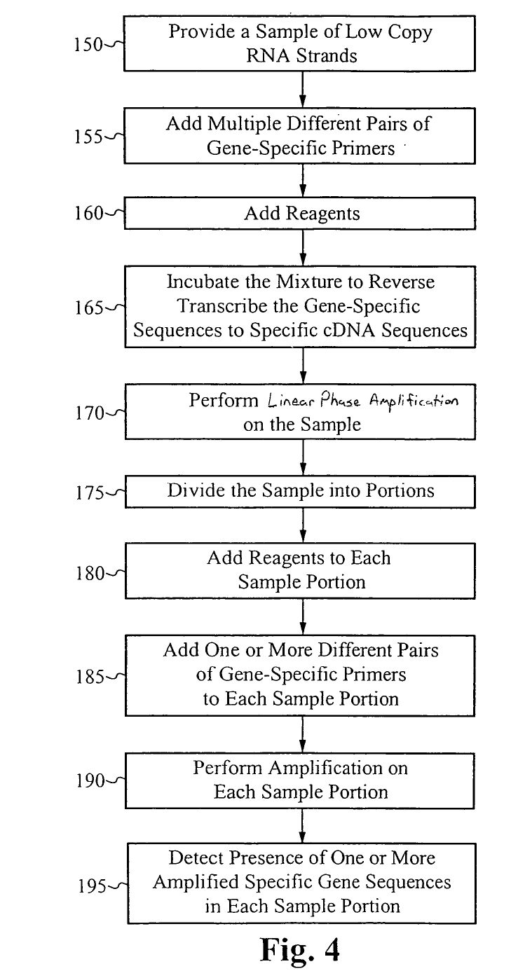

The first amplification and detection method is extendable so that it is applied to the amplification and detection of multiple different types of specific low copy targets. FIG. 4 illustrates a second amplification and detection method for detecting the presence of multiple different low copy targets. The second amplification and detection method of FIG. 4 is similar to the first amplification and detection method of FIG. 3 and includes a reverse transcription and polymerase chain reaction (RT-PCR) process. Again, only specific gene sequences are reverse transcribed in the second amplification and detection method. At the step 150, a sample is provided that includes multiple low copy RNA strands. An objective is to detect if multiple different specific gene sequences are present within the sample. At the step 155 multiple different pairs of gene-specific primers are added to the sample. In general, if the method determines the presence of ‘n’ different specific gene sequences, then ‘n’ different pairs of gene-specific primers are added. Each gene-specific primer pair corresponds to a specific gene sequence to be reverse transcribed, amplified, and eventually detected. For example, a first pair of gene-specific primers including the gene 1 RT primer corresponds to the specific gene sequence gene 1, a second pair of gene-specific primers including a gene 2 RT primer corresponds the specific gene sequence gene 2, and so on. One primer of each gene-specific primer pair is configured to bind with an RNA strand that includes the corresponding specific gene sequence such that the specific gene sequence is bound at one end by the gene-specific primer.

At the step 160, reagents used for reverse transcription and a first amplification step are added to the sample. It is understood that the step 155 and the step 160 can be combined as a single step. At the step 165, the mixture including the sample, the multiple different pairs of gene-specific primers, and the reagents, is incubated at a set temperature for a period of time. During this incubation period, a specific gene-specific primer bonds to an RNA strand if the RNA strand includes the specific gene sequence corresponding to the specific gene-specific primer. The bound gene-specific primer initiates the reverse transcription of the specific gene sequence in the RNA to a corresponding cDNA strand including the specific gene sequence. During this incubation step, each specific gene sequence present is identified and reverse transcribed into the corresponding specific gene sequence in the cDNA.

After the period of time, a first amplification step is performed on the reverse transcribed sample solution at the step 170. The first amplification step is performed by thermal cycling, or PCR. In the second amplification and detection method, the first amplification step is limited to the linear phase. Linear phase amplification is maintained during about 5-15 thermal cycles. In some embodiments, the incubation step in which reverse transcription takes place and the first amplification step are performed in a single processing chamber. Since the multiple pairs of gene-specific primers and the reagents needed for reverse transcription and the first amplification step are previously added, there is no need to add reagents and/or additional gene-specific primer pairs to the sample solution in between the incubation step and the first amplification step.

At the step 175, the first amplified sample solution is divided into sample portions. In some embodiments, the number of sample portions is equal to the number of targets (gene-specific sequences) to be detected. For example, where the amplification and detection method is configured to detect the presence of 20 different targets, the first amplified sample solution is divided into 20 sample portions. Alternatively, the sample solution is divided into fewer than the number of detected targets. At the step 180, additional reagents are added to each sample portion. In some embodiments, additional dilution can be achieved by adding a neutral solution, such as water, to one or more of the sample portions. The reagents include detection chemistries, such as TaqMan® probes. At the step 185, one or more different pairs of gene-specific primers are added to each sample portion. Each pair of gene-specific primers is targeted to a specific gene sequence. The total number of different pairs of gene-specific primers is equal to the number of targets to be detected. Each different pair of gene-specific primers added at the step 185 corresponds to the same specific gene sequence as the pair of gene-specific primers added at the step 155. For example, where the specific gene sequence is gene 2, the pair of gene-specific primers added at the step 155 and the pair of gene-specific primers added at the step 185 each target the gene 2. Each pair of gene-specific primers added at the step 185 can be the same as the corresponding pair of gene-specific primers added at the step 155. Alternatively, each pair of gene-specific primers added at the step 185 is internal to the corresponding pair of gene-specific primers added at the step 155. In the case where the number of sample portions equals the number of detected targets, one different pair of gene-specific primers is added to each sample portion. In the case where the sample solution is divided into fewer than the number of detected targets, then the different pairs of gene-specific primers are divided into groups, and one group of primer pairs is added to each sample portion.

At the step 190, a second amplification step is performed on each sample portion. As with the first amplification step, the second amplification step is performed by thermal cycling. In some embodiments, up to 40 thermal cycles are performed. Alternatively, more than 40 thermal cycles are performed. In general, the number of thermal cycles performed during the second amplification step is sufficient for entering into an exponential phase amplification and reaching saturation. The result is a sample portion including an amplified number of specific gene sequences corresponding to the specific gene-specific primer pairs added to the sample portion at the step 185. At the step 195, the amplified specific gene sequences are detected, if present, in each sample portion. Typically, during the second amplification step the detection chemistries are stimulated, such as releasing a detectable flourescent probe, which are then detected using any number of conventional detection means.

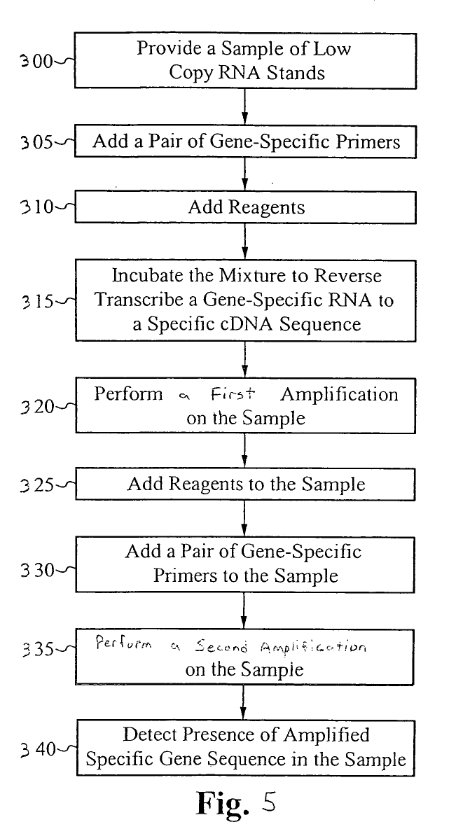

FIG. 5 illustrates a third amplification and detection method for detecting the presence of a low copy target. The third amplification and detection method is a modification of the first amplification and detection method. In the third amplification and detection method, the first amplification step is no longer restricted to the linear phase and the number of thermal cycles performed during the second amplification step is reduced to a minimum number sufficient to generate a detectable level of the target. In this manner, fewer thermal cycles are necessary to generate a detectable signal.

At the step 300, a sample is provided that includes a low copy RNA strand. An objective is to detect if a specific gene sequence in the RNA is present within the sample. At the step 305 a pair of gene-specific primers are added to the sample. By way of example, the specific gene sequence that is to be targeted and amplified is referred to as gene 1. In this case, the pair of gene-specific primers are specific to gene 1. One of the pair of gene-specific primers, referred to as the gene I reverse transcription (RT) primer, is configured to bond with the RNA strand at one end of the gene. At the step 310, reagents are added to the sample. It is understood that the step 305 and the step 310 can be combined as a single step. The third amplification and detection method combines a reverse transcription step and a first amplification step within a single processing chamber. The pair of gene-specific primers including the gene 1 RT primer, reagents for the reverse transcription step, and reagents for the first amplification step are all added to the original sample prior to performing the reverse transcription step. At the step 315, the mixture including the sample, the pair of gene-specific primers, and the reagents, is incubated at a first temperature for a first period of time. During this incubation period, the gene 1 RT primer binds to an RNA strand if the RNA strand includes the gene 1. The bound gene 1 RT primer initiates the reverse transcription of the gene 1 in the RNA to a corresponding cDNA sequence, including the gene 1. During this incubation step, the gene-specific sequence in the RNA is identified and reverse transcribed into the corresponding specific cDNA sequence.

After the first period of time, a first amplification step is performed on the reverse transcribed sample solution at the step 320. The first amplification step is performed by thermal cycling, or PCR. The number of thermal cycles performed during the first amplification step is sufficient to move beyond the linear phase of amplification and into the exponential phase. However, the number of thermal cycles performed during the first amplification stage is less than the number of thermal cycles sufficient to reach saturation. Saturation is the point where additional cycles do not increase the yield of specific product. For example, the first amplification step is performed for 20-25 cycles. In some embodiments, the incubation step in which reverse transcription takes place and the first amplification step are performed in a single processing chamber. Since the pair of gene-specific primers and the reagents needed for reverse transcription and amplification are previously added, there is no need to add reagents and/or additional primers to the sample solution in between the incubation step and the first amplification step.

At the step 325, additional reagents are added to the first amplified sample solution, thereby diluting the first amplified sample solution. In some embodiments, an additional dilution step is performed whereby an additional neutral solution, such as water, is added in addition to the reagents added in the step 325. The reagents include detection chemistries, such as TaqMan® probes. At the step 330, a pair of gene-specific primers targeted to the gene I is added to the first amplified sample solution. The pair of gene-specific primers added at the step 330 can be the same as the pair of gene-specific primers added at the step 305. Alternatively, the pair of gene-specific primers added at the step 330 are not the same as the pair of gene-specific primers added at the step 305. Instead, each of the pair of gene-specific primers added in the step 330 are internal to the pair of gene-specific primers added at the step 305. In general, the pair of gene-specific primers added at the step 330 is equal to or internal to the pair of gene-specific primers added at the step 305.

At the step 335, a second amplification step is performed on the first amplified sample solution. As with first amplification step, the second amplification step is performed by thermal cycling. In some embodiments, 20-25 thermal cycles are performed. Alternatively, more or less than 20-25 thermal cycles are performed. In general, the number of thermal cycles performed during the second amplification step is a minimum number sufficient to generate a detectable level of the copy target (e.g. each gene-specific sequence being targeted). In most applications, this minimum number of thermal cycles is insufficient to reach saturation. An advantage of the third amplification and detection method is the elimination of non-specific product generated during the second amplification step. The result is a sample solution including an amplified number of cDNA strands including the gene-specific sequence. At the step 340, the amplified specific gene sequence is detected, if present. Typically, during the second amplification step the detection chemistries are stimulated, such as releasing a detectable flourescent probe, which are then detected using any number of conventional detection means.

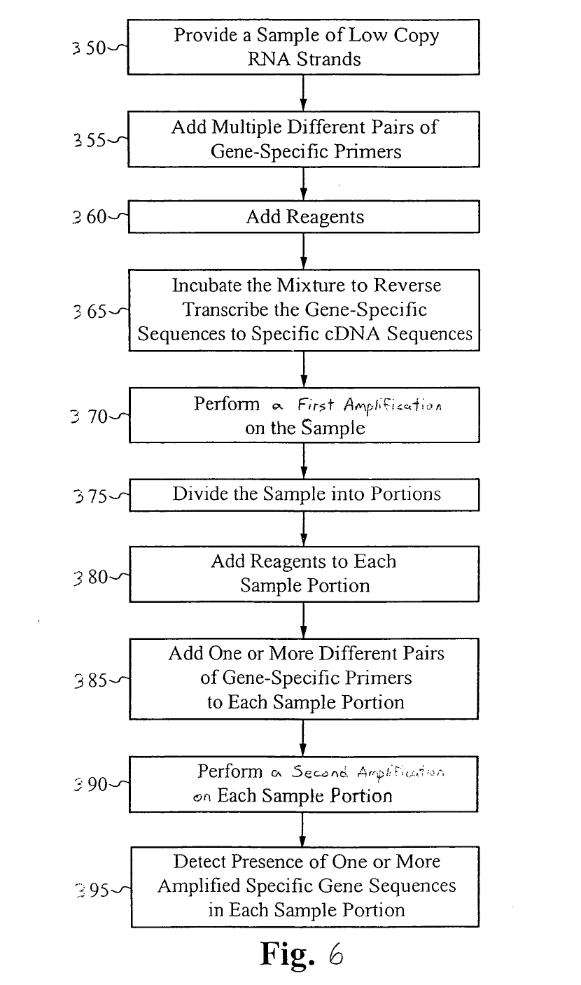

FIG. 6 illustrates a fourth amplification and detection method for detecting the presence of multiple different low copy targets. The fourth amplification and detection method is a modification of the second amplification and detection method. In the fourth amplification and detection method, the first amplification step is no longer restricted to the linear phase and the number of thermal cycles performed during the second amplification step is reduced to a minimum number sufficient to generate a detectable level of the copy target. In this manner, fewer thermal cycles are necessary to generate a detectable signal.

At the step 350, a sample is provided that includes multiple low copy RNA strands. An objective is to detect if multiple different specific gene sequences are present within the sample. At the step 355 multiple different pairs of gene-specific primers are added to the sample. At the step 360, reagents used for reverse transcription and amplification are added to the sample. It is understood that the step 355 and the step 360 can be combined as a single step. At the step 365, the mixture including the sample, the multiple different pairs of gene-specific primers, and the reagents, is incubated at a set temperature for a period of time. During this incubation period, a specific gene-specific primer bonds to an RNA strand if the RNA strand includes the specific gene sequence corresponding to the specific gene-specific primer. The bound gene-specific primer initiates the reverse transcription of the specific gene sequence in the RNA to a corresponding cDNA strand including the specific gene sequence. During this incubation step, each specific gene sequence present is identified and reverse transcribed into the corresponding specific gene sequence in the cDNA.

After the period of time, a first amplification step is performed on the reverse transcribed sample solution at the step 370. The first amplification step is performed by thermal cycling, or PCR. The number of thermal cycles performed during the first amplification step is sufficient to move beyond the linear phase of amplification and into the exponential phase. However, the number of thermal cycles performed during the first amplification stage is less than the number of thermal cycles sufficient to reach saturation. For example, the first amplification step is performed for 20-25 cycles. In some embodiments, the incubation step in which reverse transcription takes place and the first amplification step are performed in a single processing chamber. Since the multiple pairs of gene-specific primers and the reagents needed for reverse transcription and the first amplification step are previously added, there is no need to add reagents and/or additional gene-specific primer pairs to the sample solution in between the incubation step and the first amplification step.

At the step 375, the first amplified sample solution is divided into sample portions. In some embodiments, the number of sample portions is equal to the number of targets (gene-specific sequences) to be detected. Alternatively, the sample solution is divided into fewer than the number of detected targets. At the step 380, additional reagents are added to each sample portion. In some embodiments, additional dilution can be achieved by adding a neutral solution, such as water, to one or more of the sample portions. The reagents include detection chemistries, such as TaqMan® probes. At the step 385, one or more different pairs of gene-specific primers are added to each sample portion. Each pair of gene-specific primers is targeted to a specific gene sequence. The total number of different pairs of gene-specific primers is equal to the number of targets to be detected. Each different pair of gene-specific primers added at the step 385 corresponds to the same specific gene sequence as the pair of gene-specific primers added at the step 355. For example, where the specific gene sequence is gene 2, the pair of gene-specific primers added at the step 355 and the pair of gene-specific primers added at the step 385 each target the gene 2. Each pair of gene-specific primers added at the step 385 can be the same as the corresponding pair of gene-specific primers added at the step 355. Alternatively, each pair of gene-specific primers added at the step 385 is internal to the corresponding pair of gene-specific primers added at the step 355. In the case where the number of sample portions equals the number of detected targets, one different pair of gene-specific primers is added to each sample portion. In the case where the sample solution is divided into fewer than the number of detected targets, then the different pairs of gene-specific primers are divided into groups, and one group of primer pairs is added to each sample portion.

At the step 390, a second amplification step is performed on each sample portion. As with the first amplification step, the second amplification step is performed by thermal cycling. In some embodiments, 20-25 thermal cycles are performed during the second amplification step. Alternatively, more or less than 20-25 thermal cycles are performed. In general, the number of thermal cycles performed during the second amplification step is a minimum number sufficient to generate a detectable level of the copy target (e.g. each specific gene sequence being targeted). In most applications, this minimum number of thermal cycles is insufficient to reach saturation. An advantage of the fourth amplification and detection method is the elimination of non-specific product generated during the second amplification step. The result is a sample portion including an amplified number of specific gene sequences corresponding to the specific gene-specific primer pairs added to the sample portion at the step 385. At the step 395, the amplified specific gene sequences are detected, if present, in each sample portion.

Typically, during the second amplification step the detection chemistries are stimulated, such as releasing a detectable flourescent probe, which are then detected using any number of conventional detection means.

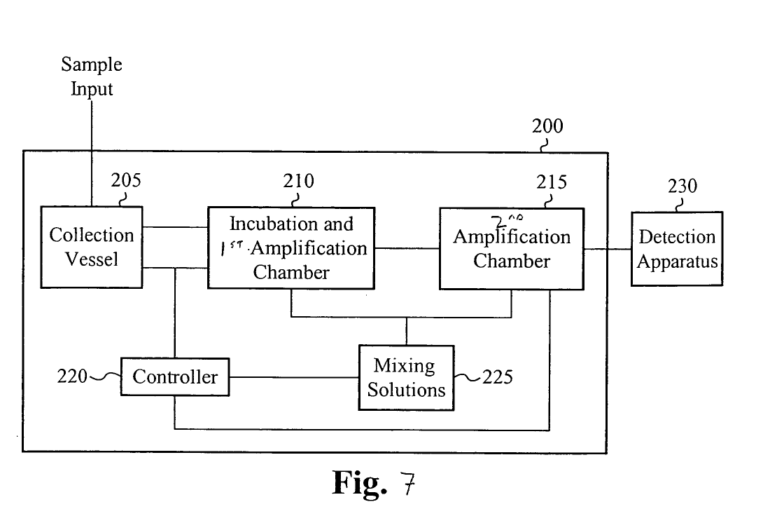

In some embodiments, the amplification and detection method is implemented within an amplification and detection apparatus. FIG. 7 illustrates a functional block diagram of an exemplary amplification and detection apparatus configured to implement the amplification and detection method. It is understood that the configuration of the amplification and detection apparatus shown in FIG. 7 and described below is for exemplary purposes only. The amplification and detection apparatus includes an amplification apparatus 200 and a detection apparatus 230. As shown in FIG. 7, the amplification apparatus 200 is configured as a stand-alone device and is coupled to the detection apparatus 230. Alternatively, the amplification apparatus and the detection apparatus are integrated within a single device.

The amplification apparatus 200 includes a collection vessel 205, an incubation and first amplification chamber 210, a second amplification chamber 215, and a mixing solutions module 225 coupled together via microfluidic circuitry. The amplification apparatus 200 also includes a controller 220 coupled to control the operation of the microfluidic circuitry, the collection vessel 205, the incubation and first amplification chamber 210, the second amplification chamber 215, and the mixing solutions module 225. The controller 220 also controls fluid flow into and out of the amplification apparatus 200. In some embodiments, the controller 220 is also coupled to control the operation of the detection apparatus 230.

The collection vessel 205 is configured to receive the input sample. The amplification and detection apparatus is configured to amplify and detect the presence of one or more low copy targets that might be included in the input sample. The sample is then directed to the incubation and first amplification chamber 210. The incubation and first amplification chamber 210 is configured to perform the incubation and first amplification steps described above. The mixing solutions module 225 is coupled to the incubation and first amplification chamber 210 to provide the one or more different pairs of gene-specific primers and the reagents necessary to perform the incubation and first amplification steps. The mixing solutions module 225 includes multiple containment vessels for storing the gene-specific primers and the reagents. The various primers and reagents can be stored independently or in any pre-mixed combination. In some embodiments, the incubation and first amplification chamber 210 is the collection vessel 205, and the input sample is provided to the incubation and first amplification chamber 210.

The incubation and first amplification chamber 210 outputs the first amplified sample solution to the second amplification chamber 215. The second amplification chamber 215 is configured to perform the second amplification step described above. Where the amplification and detection method is configured to divide the first amplified sample solution into sample portions, the microfluidic circuitry is configured to divide the first amplified sample solution and to provide each sample portion to the second amplification chamber 215. In this case, the second amplification chamber 215 includes multiple separate amplification chambers, each to receive a sample portion. Alternatively, the amplification and detection apparatus includes a metering and distribution module that is configured to divide the first amplified sample solution into the sample portions.

The mixing solutions module 225 is coupled to the second amplification chamber 215 to provide the one or more different pairs of gene-specific primers and the reagents necessary to perform the second amplification step. The second amplification chamber 215 outputs the second amplified sample solution, or each second amplified sample portion, to the detection apparatus 230. The detection apparatus 230 is configured to detect if one or more specific targets are present within the second amplified sample solution, or within each of the second amplified sample portions.

In some embodiments, the amplification and detection apparatus is configured to automatically perform one, some, or all of the steps of the amplification and detection method described above. In this manner, one, some, or all of the steps of the amplification and detection method can be automated.

The amplification and detection apparatus can be implemented as a stand-alone device or as part of a larger system, such as a particle collection and detection system described in the co-owned and co-pending U.S. Patent Application Serial No. (MFSI-00700), entitled “An Integrated Substance Collection and Detection System,” which is hereby incorporated by reference.

The amplification and detection method includes a two-step RT-PCR approach for detection of multiple low-abundance targets. This method requires significantly less starting material than conventional approaches. Such improved sensitivity allows detection in small samples, such as 1-100 cells. In some embodiments, detection of one or more low copy targets is achieved via real-time PCR using “RT-amplification” product generated by controlled hot start multiplex RT-PCR. In contrast with conventional approaches, such as TaqMan®, this method requires lower amounts of starting material and therefore can be applied to detect multiple low expressed targets in environmental samples. The amplification and detection method allows simultaneous quantification of hundreds of targets using as little as 2.5 fg of sample, whereas conventional assays require substantially larger amounts of nucleic acid, usually from 10 ng to 1 ug per reaction. Conventional gene microarrays require even larger amounts of starting RNA (1-10 ug), and individual gene probes in labeled complex cDNA/cRNA mixtures may have unique secondary structures, melting temperatures, and reassociation rates which makes hybridization of all gene probes under optimum condition nearly impossible. At this time, no other technique offers the potential for simultaneous rapid, accurate, and reliable quantification of multiple targets in small samples (1-100 cells).

The amplification and detection methods are described above as performing an amplification method on a sample that includes one or more different RNA sequences. In some embodiments, the initial sample also includes one or more different DNA sequences. In other embodiments, the sample may not include any RNA sequences, but instead include only one or more different DNA sequences. In such cases, any DNA sequences present in the sample during the reverse transcription step remain unchanged. For example, if a sample includes both RNA sequences and DNA sequences, or just DNA sequences, when the incubation step is performed whereby gene-specific RNA sequences are reverse transcribed into corresponding cDNA sequences, this process has no impact on the DNA sequences included within the sample. In the case where both RNA sequences and DNA sequences are present within the sample, the gene-specific RNA sequences are reverse transcribed, while the DNA sequences remain unaffected. In the case where only DNA sequences are present in the sample, the incubation step does not yield any results, since no RNA sequences are present. In either case, this eliminates the need to first determine if the sample includes RNA sequences, DNA sequences, or both, and then subsequently having to alter the process according to that knowledge. During the first amplification step and the second amplification step, any original DNA sequences that correspond to the gene-specific primers are amplified, similar to any gene-specific reverse transcribed cDNA sequences as described above.

The present invention has been described in terms of specific embodiments incorporating details to facilitate the understanding of the principles of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

What is claimed is:

1. A method of detecting multiple limited copy targets, the method comprising:
a. providing a sample including a plurality of differently sequenced RNA strands;
b. adding multiple different first pairs of gene-specific primers to the sample, wherein each first pair of gene-specific primers corresponds to a specific gene sequence;
c. adding reagents to the sample;
d. incubating the sample at a first temperature for a first period of time such that if one or more of the specific gene sequences are present within the sample, a corresponding gene-specific primer adheres to each specific RNA strand that includes one of the present specific gene sequences, thereby reverse transcribing each specific RNA strand to a corresponding cDNA strand including the specific gene sequence;
e. performing a first thermal cycling process to amplify each specific gene sequence within the cDNA strand according to a linear phase amplification;
f. dividing the sample into portions;
g. adding reagents and one or more different second pairs of gene-specific primers to each sample portion, wherein each second pair of gene-specific primers corresponds to one of the specific gene sequences;
h. performing a second thermal cycling process on each sample portion to amplify the one or more specific gene sequences in each sample portion; and
i. detecting the presence of the one or more specific gene sequences in each sample portion.

2. The method of claim 1 wherein N different pairs of gene-specific primers are added to the sample such that the presence of up to N specific gene sequences are detected.

3. The method of claim 2 wherein the sample is divided into N portions and one second pair of gene-specific primers is added to each sample portion.

4. The method of claim 1 wherein a minimum quantity of the sample provided is greater than or equal to about 2.5 femtograms.

5. The method of claim 1 wherein the second thermal cycling process is performed to saturation.

6. The method of claim 1 wherein performing the first thermal cycling process is automatically performed after the first period of time.

7. The method of claim 1 wherein incubating the sample and performing the first thermal cycle are performed within a single containment vessel.

8. The method of claim 1 wherein the first thermal cycling process and the second thermal cycling process each comprise a polymerase chain reaction.

9. The method of claim 1 wherein the first thermal cycling process includes five to fifteen cycles.

10. The method of claim 1 wherein the second thermal cycling process includes at least a number of thermal cycles sufficient for entering into an exponential phase of amplification.

11. A method of detecting a limited copy target, the method comprising:
a. providing a sample including one or more differently sequenced RNA strands;
b. adding a first pair of gene-specific primers to the sample, wherein the first pair of gene-specific primers corresponds to a specific gene sequence;
c. adding reagents to the sample;
d. incubating the sample at a first temperature for a first period of time such that if the specific gene sequence is present within the sample, one of the first pair of gene-specific primers adheres to a specific RNA strand that includes the specific gene sequence, thereby reverse transcribing the specific RNA strand to a corresponding cDNA strand including the specific gene sequence;
e. performing a first thermal cycling process to amplify the specific gene sequence within the cDNA strand according to a linear phase amplification;
f. adding reagents and a second pair of gene-specific primers to the sample, wherein the second pair of gene-specific primers correspond to the specific gene sequence;
g. performing a second thermal cycling process to amplify the specific gene sequence; and
h. detecting the presence of the specific gene sequence.

12. The method of claim 11 wherein performing the first thermal cycling process is automatically performed after the first period of time.

13. The method of claim 11 wherein incubating the sample and performing the first thermal cycle are performed within a single containment vessel.

14. The method of claim 11 wherein the first thermal cycling process and the second thermal cycling process each comprise a polymerase chain reaction.

15. The method of claim 11 wherein the first thermal cycling process includes five to fifteen cycles.

16. The method of claim 11 wherein the second thermal cycling process includes at least a number of thermal cycles sufficient for entering into an exponential phase of amplification.

17. The method of claim 11 further comprising diluting the sample with a neutral solution prior to performing the second thermal cycling process.

18. The method of claim 11 wherein the second thermal cycling process is performed to saturation
